(19) United States
(12) Patent Application Publication
(10) Pub. No.: US 2010/0009409 A1
(43) Pub. Date: Jan. 14, 2010

(54) MOLECULAR VARIANT FIBRINOGEN FUSION PROTEINS

(75) Inventors: Jeffrey A. Hubbell, Morges (CH); Thomas H. Barker, Atlanta, GA (US)

Correspondence Address: Pabst Patent Group LLP, 1545 PEACHTREE STREET NE, SUITE 320, ATLANTA, GA, 30309, US

(73) Assignee: Ecole Polytechnique Federale de Lausanne, Lausanne (CH)

(21) Appl. No.: 11/997,010

(22) PCT Filed: Jul. 14, 2006
(86) PCT No.: PCT/US06/27559

§ 371 Date: Jun. 26, 2008

Related U.S. Application Data
(60) Provisional application No. 60/704,075, filed on Jul. 29, 2005.

(51) Int. Cl. C12P 21/02; C07H 21/04; C12N 15/74; C07K 14/75

(52) U.S. Cl. 435/69.6; 536/23.5; 435/320.1; 530/382

(57) ABSTRACT

Fibrinogen fusion proteins, methods of making, and methods of using fibrinogen fusion proteins are described. In a preferred embodiment the fibrinogen fusion protein contains a truncated Aα chain of fibrinogen. The Aα chain contains truncation site, which is a deletion of amino acids at its C-terminal region. A non-fibrinogen protein or peptide is C-terminally attached to the truncation site. The fibrinogen fusion proteins can be used alone or mixed with native fibrinogen to form fibrin polymer.

### CROSS-REFERENCE TO RELATED APPLICATIONS

This application claims the benefit of U.S. Ser. No. 60/704,075 filed in the U.S. Patent and Trademark Office on Jul. 29, 2005, by Jeffrey A. Hubbell and Thomas A. Barker.

### FIELD OF THE INVENTION

The present application is directed to variants of fibrinogen and their use for incorporating proteins or peptides into a fibrin polymer which can be used for drug delivery or in tissue engineering.

### BACKGROUND OF THE INVENTION

Fibrinogen is a highly evolutionarily-conserved, soluble serum protein that serves as the source of fibrin in blood to form clots that are critical to hemostasis, which is the ability of the body to control and maintain adequate blood flow after injury to the vascular system. The extensively studied human fibrinogen is a 340,000 dalton protein, which has a complex oligomeric structure that contains three pairs of related polypeptide chains, designated (Aα)₂, (Bβ)₂, and γ₂ polypeptide chains. Chemical structural analysis and electron microscopy have demonstrated that the protein has a trinodular structure. In particular, two Aα Bβ and γ subunits are oriented in an anti-parallel configuration. The amino terminal portions of the six chains are bundled together in a central “E” domain. Two coiled-coil strands extend outward from either side of the E domain to two terminal nodes, the “D” domains. These coiled-coil regions are 110 amino acids long and composed of all three chains. The D domains contain two high affinity Ca²⁺ binding sites and are involved with the E domain in fibrin polymerization. Extensive disulfide bridges covalently cross-link the two subunits and stabilize the globular domains. The carboxy-terminal portions of the Aα chains form flexible extensions beyond the D domains. The D domain contains Factor XIIIa crosslinking sites and is the primary site of plasmin digestion during fibrinolysis. The individual polypeptide chains of human fibrinogen are extensively linked by disulfide bonds to form an elongated dimeric molecule (for reviews, see, e.g., Hawiger, Semin Hematol, 32:99-109 (1995); Doolittle et al., FASEB J, 10:1464-1470 (1996)).

Fibrin is a natural gel with several biomedical applications. Fibrin gel has been used as a sealant because of its ability to bind to many tissues and its natural role in wound healing. Some specific applications include use as a sealant for vascular graft attachment, heart valve attachment, bone positioning in fractures and tendon repair (Sierra, D. H., Journal of Biomaterials Applications, 7:309-352, 1993). Additionally, these gels have been used as drug delivery devices, and for neuronal regeneration (Williams, et al., Journal of Comparative Neurobiology, 264:284-290, 1987). Although fibrin does provide a solid support for tissue regeneration and cell ingrowth, there are few active sequences in the monomer that directly enhance these processes.

The process by which fibrinogen is polymerized into fibrin has also been characterized. Initially, a protease cleaves the dimeric fibrinogen molecule at the two symmetric sites. There are several possible proteases than can cleave fibrinogen, including thrombin, reptilase, and protease III, and each one severs the protein at a different site (Francis, et al., Blood Cells, 19:291-307, 1993). For example, thrombin cleaves at the Arg16-Arg17 bond in the Aα chains and at the Arg14-Gly15 bond on the Bβ chains of fibrinogen. Once the fibrinogen is cleaved, a self-polymerization step occurs in which the fibrinogen monomers come together and form a non-covalently crosslinked polymer gel (Sierra, 1993). This self-assembly happens because binding sites become exposed after protease cleavage occurs. Once they are exposed, these binding sites in the center of the molecule can bind to other sites on the fibrinogen chains, which are present at the ends of the peptide chains (Stryer, L. In Biochemistry, W.H. Freeman & Company, NY, 1975). In this manner, a polymer network is formed. Factor XIIIa, a transglutaminase activated from Factor XIII by thrombin proteolysis, may then covalently crosslink the polymer network. Other transglutaminases exist and may also be involved in covalent crosslinking and grafting to the fibrin network.

Once a crosslinked fibrin gel is formed, the subsequent degradation is tightly controlled. One of the key molecules in controlling the degradation of fibrin is 2-plasmin inhibitor (Aoki, N., Progress in Cardiovascular Disease, 21:267-286, 1979). This molecule acts by crosslinking to the chain of fibrin through the action of Factor XIIIa (Sakata, et al., Journal of Clinical Investigation, 65:290-297, 1980). By attaching itself to the gel, a high concentration of inhibitor can be localized to the gel. The inhibitor then acts by preventing the binding of plasminogen to fibrin (Aoki, et al., Thrombosis and Haemostasis, 39:22-31, 1978) and inactivating plasmin (Aoki, 1979). The 2-plasmin inhibitor contains a glutamine substrate. The exact sequence has been identified as NQEQVSPL (SEQ ID NO: 1), with the first glutamine being the active amino acid for crosslinking.

The components required for making fibrin gels can be obtained in two ways. One method is to cryoprecipitate the fibrinogen from plasma, in which Factor XIII precipitates with the fibrinogen. The proteases are purified from plasma using similar methods. Another technique is to make recombinant forms of these proteins either in culture or with transgenic animals. The advantage of this is that the purity is much higher, and the concentrations of each of these components can be controlled.

Current methods for incorporation of a drug to be delivered or incorporated within the fibrin include cross-linking of the drug to the fibrinogen, and physical incorporation into a fibrin matrix. The latter is difficult to control, however, with variable incorporation as well as release, and the former may interfere with fibrin crosslinking to form a gel.

Therefore, it is an object of the present invention to provide methods of making fibrinogen fusion proteins to enhance the incorporation of a therapeutic protein or peptide species into a fibrin polymer.

### BRIEF SUMMARY OF THE INVENTION

Fibrinogen fusion proteins and methods of making fibrinogen fusion proteins are described. The fibrinogen fusion proteins can be mixed with carrier proteins that serve a protective role or mixed with proteins that interact with the fusion protein in a specific way (e.g., DNA is mixed with a DNA-binding fibrinogen fusion protein). The fibrinogen fusion proteins can be used alone or mixed with native fibrinogen to form fibrin polymer. In a preferred embodiment the fibrinogen fusion protein contains a truncated Aα chain of fibrinogen. The Aα chain, which normally consists of amino acids 1 to 644, contains a truncation site, which is a deletion of amino acids at its C-terminal region. Preferably, the truncated Aα chain of fibrinogen consists of amino acids 1 to 189, more preferably the truncated Aα chain of fibrinogen consists of amino acids 1 to 184, and most preferably the truncated Aα chain of fibrinogen consists of amino acids 1 to 180. It should be understood that any number of possible deletions can be made to the Aα chain of fibrinogen, so long as this molecular modification takes place C-terminally to amino acid 179. Amino acids 1 to 179 of the Aα chain are required in order for mature fibrinogen and fibrin polymers to form.

In a preferred embodiment, a non-fibrinogen protein or peptide is C-terminally attached to the truncation site. Representative non-fibrinogen proteins that can be incorporated include, but are not limited to, adhesion proteins, growth factors, cytokines, chemokines, antiadhesion proteins, immunostimulatory proteins, immunomodulatory proteins, protein-binding proteins, nucleic acid-binding proteins, heparin-binding proteins, virus-binding proteins, cytotoxic proteins, enzymatically active proteins, and protease inhibitors. Domains or peptide portions of proteins can also be inserted into the truncation site of the fibrinogen Aα chain.

In a preferred embodiment, the modified fibrinogen fusion proteins are produced by transfection of a vector encoding the Aα chain fusion protein, the Bβ chain and the γ chain of fibrinogen or co-transfection of vectors encoding each chain separately, into a host cell such as a bacterial, yeast, insect cell, or mammalian cell. The fibrinogen fusion proteins are then expressed and isolated from these cells.

### BRIEF DESCRIPTION OF THE DRAWINGS

FIG. 1 is an illustration of the structure of fibrinogen (Yang, et al., Biochemistry 40:12515-12523 (2001)).

FIG. 2 is an illustration of an exemplary fibrinogen fusion protein.

FIG. 3 is a schematic diagram of an exemplary cloning strategy.

FIG. 4 is a schematic diagram of an exemplary method for generating fibrinogen Aα fusion protein products.

### DETAILED DESCRIPTION OF THE INVENTION

### I. Modified Fibrinogen Fusion Proteins

A. Truncated Fibrinogen

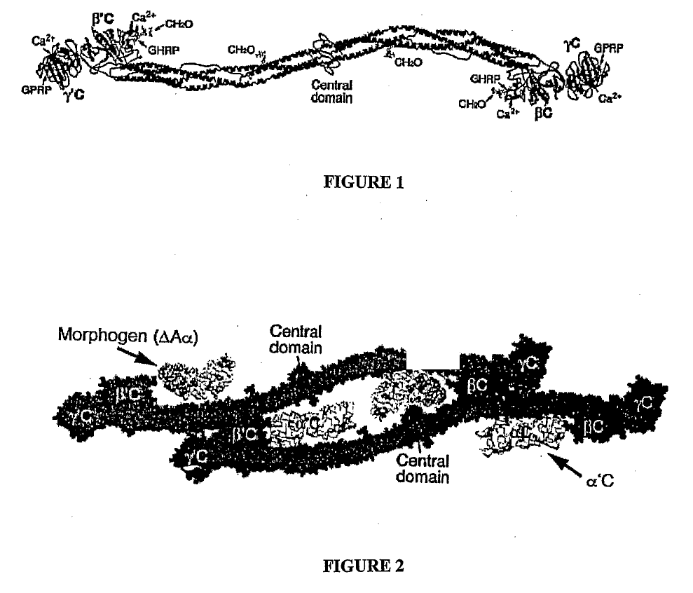

It has been discovered that fibrinogen, which is a homodimer of a heterotrimer (Aα, Bβ, and γ chains), and its native structure and function can be modified at the carboxy-termini of the Aα individual chains to produce a truncated fibrinogen or fibrinogen fusion protein. Neither any of the amino-termini nor the carboxy-termini of the Bβ and γ chains can be modified since polymer formation requires both close association between Bβ and γ chains of adjacent fibrin monomers and direct binding (via “A and B holes”) to sites on the amino-termini of Aα and Bβ chains exposed by thrombin activation (a.k.a. “α and β knobs”). The structure of fibrinogen is illustrated in FIG. 1.

As used herein, “fibrinogen” refers to the homodimer of a heterotrimer, preferably of human origin, and variants thereof including conservative substitutions, additions, and deletions therein (other than of the carboxyl region) not affecting the native structure or function. In the preferred embodiment, the fibrinogen is native human fibrinogen terminated at the carboxyl domain as described below. Included within the scope of the present invention are, deglycosylated or unglycosylated derivatives of such fibrinogen proteins, and biologically active amino acid sequence variants of fibrinogen, including alleles, and in vitro generated covalent derivatives of fibrinogen proteins that demonstrate fibrinogen protein activity.

Amino acid sequence variants of fibrinogen fall into one or more of three classes: substitutional, insertional or deletional variants. Insertions include amino and/or carboxyl terminal fusions as well as intrasequence insertions of single or multiple amino acid residues. Fibrinogen fusion proteins include, for example, hybrids of mature fibrinogen with polypeptides that are homologous with fibrinogen, for example, in the case of human fibrinogen, secretory leaders from other secreted human proteins. Fibrinogen also include hybrids of fibrinogen with polypeptides homologous to the host cell but not to fibrinogen, as well as, polypeptides heterologous to both the host cell and fibrinogen. Fusions within the scope of this invention are amino or carboxy terminal fusions with either prokaryotic peptides or signal peptides of prokaryotic, yeast, viral or host cell signal sequences.

Insertions can also be introduced within the mature coding sequence of fibrinogen. These, however, ordinarily will be smaller insertions than those of amino or carboxyl terminal fusions, on the order of 1 to 4 residues. Unless otherwise states, representative fibrinogen variations described herein are variations in the mature fibrinogen sequence; they are not pre-fibrinogen variants.

Insertional amino acid sequence variants of fibrinogen are those in which one or more amino acid residues are introduced into a predetermined site in the target fibrinogen. Most commonly insertional variants are fusions of heterologous proteins or polypeptides of the amino or carboxyl terminus of fibrinogen. Immunogenic fibrinogen derivatives are made by fusing a polypeptide sufficiently large to confer immunogenicity to the target sequence by cross-linking in vitro or by recombinant cell culture transformed with DNA encoding the fusion. Such immunogenic polypeptides can be bacterial polypeptides such as trpLE, beta-galactosidase and the like.

Deletion variants are characterized by the removal of one or more amino acid residues from the fibrinogen protein sequence. These variants ordinarily are prepared by site specific mutagenesis of nucleotides in the DNA encoding the fibrinogen, thereby producing DNA encoding the variant, and thereafter expressing the DNA in recombinant cell culture. However, variant fibrinogen protein fragments may be conveniently prepared by in vitro synthesis. The variants typically exhibit the same qualitative biological activity as the naturally-occurring analogue, although variants also are selected in order to modify the characteristics of fibrinogen.

While the site for introducing an amino acid sequence variation is predetermined, the mutation per se need not be predetermined. For example, in order to optimize the performance of a mutation at a given site, random mutagenesis may be conducted at the target codon or region and the expressed fibrinogen variants screened for the optimal combination of desired activity. Techniques for making substitution mutations at predetermined sites in DNA having a known sequence are well known, for example M13 primer mutagenesis.

Amino acid substitutions are typically of single residues; insertions usually will be on the order of about from 1 to 10 amino acid residues; and deletions will range about from 1 to 30 residues. Deletions of insertions preferably are made in adjacent pairs; i.e. a deletion of 2 residues or insertion of 2 residues. Substitutions, deletion, insertions or any combination thereof may be combined to arrive at a final construct. Obviously, the mutations that will be made in the DNA encoding the variant fibrinogen must not place the sequence e out of reading frame and preferably will not create complementary regions that could produce secondary mRNA structure.

Substitutional variants are those in which at least one residue sequence has been removes and a different residue inserted in its place. Substitutional changes in function or immunological identity can be made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in fibrinogen protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or praline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for 9 or by) one not having a side chain, e.g., glycine.

Substitutional or deletional mutagenesis can be employed to eliminate N- or O-linked glycosylation sites (e.g. by deletion or substitution of asparaginyl residues in Asn-X-Thr glycosylation sites). Alternatively, unglycosylated fibrinogen can be produced in recombinant prokaryotic cell culture. Deletions or substitutions of cysteine or other labile residues also may be desirable, for example in increasing the oxidative stability or selecting the preferred disulfide bond arrangement of the fibrinogen. Deletions or substitutions of potential proteolysis sites, e.g. Arg Arg, are accomplished for example by deleting one of the basic residues or substituting one by glutaminyl or histidyl residues.

A DNA isolate is understood to mean chemically synthesized DNA, cDNA or genomic DNA with or without the 3′ and/or 5′ flanking regions. DNA encoding fibrinogen can be obtained from other sources than humans by a) obtaining a cDNA library from the tissue containing the fibrinogen mRNA of a particular animal, b) conducting hybridization analysis with labeled DNA encoding human fibrinogen or fragments thereof (usually, greater than 100 bp) in order to detect clones in the cDNA library containing homologous sequences, and c) analyzing the clones by restriction enzyme analysis and nucleic acid sequencing to identify full-length clones.

The carboxy-terminus of the Aα chain of fibrinogen presents the best possible location for fusion modification due to its relative inactivity in both protein assembly and in the process of fibrin polymer formation. The carboxy-terminal region of the fibrinogen Aα chain is not required for bioassembly of the mature protein, nor does it participate in the formation of polymer. Evidence to this effect comes from polymerization studies on fibrinogen with selected cleavage of the C-terminal Aα sequences of fibrinogen. This cleaved product was capable of forming polymer in turbidity assays. Furthermore this cleaved product was also capable of undergoing intermolecular crosslinking through the action of the transglutaminase Factor XIII. The fact that fibrinogen (a highly evolutionarily-conserved protein) from chicken lacks the C-terminal region of Aα chain (Yang, et al., Biochemistry 40:12515-12523 (2001)) and a truncation mutant of human fibrinogen, called “fibrinogen Aα 251,” (Gorkun, et al., Biochemistiy 37:15434-15441 (1998)) also lacks a significant portion of this domain, yet are fully assembled and active indicate that there are regions of this polypeptide chain that can be modified with little effect on the polymer forming function of the molecule. As described in more detail below, both simple additions to the carboxy-terminal end of full length Aα chain, as well as fusions to genetically engineered truncation mutations are useful. In the preferred embodiment, truncation mutations are designed to include the amino-terminal domain of Aα chain extending through the second disulfide ring structure of the so called “coiled coil” region of fibrinogen, which are amino acids 1 through 179 of the Homo sapiens fibrinogen Aα chain. It should be understood that any number of possible deletions can be made to the Aα chain to accommodate any number of insertions and substitutions, so long as this molecular modification takes place C-terminally to amino acid 179. It should also be understood that the amino acids comprising the amino-terminal domain of Aα chain extending through the second disulfide ring structure of the so called “coiled coil” region of fibrinogen derived from other species may be determined by sequence alignment about Cys 179 in the Homo sapiens sequence. Naturally occurring truncation mutations in Homo sapiens which do not include the Ac: chain sequence through this critical disulfide ring structure have been shown to result in dysfibrinogenemia, a condition characterized by improper or absent fibrin formation.

In a preferred embodiment, the Aα chain, which normally consists of amino acids 1 to 644, contains a truncation site, which is a deletion of amino acids at its C-terminal region. Preferably, the truncated Aα chain of fibrinogen consists of amino acids 1 to 189, more preferably the truncated Aα chain of fibrinogen consists of amino acids 1 to 184, and most preferably the truncated Aα chain of fibrinogen consists of amino acids 1 to 180. It should be understood that any number of possible deletions can be made to the Aα chain of fibrinogen, so long as this molecular modification takes place C-terminally to amino acid 179. Amino acids 1 to 179 of the Aα chain are required in order for mature fibrinogen and fibrin polymers to form.

B. Bioactive Factors for Incorporation into Truncated Fibrinogen.

As described herein, X is indicative of a bioactive factor that can be C-terminally attached to the truncated fibrinogen Aα chain. The choice of X depends in part upon the desired application. The peptide or protein domain X is inserted in the space that is created at the C-terminus of the fibrinogen Aα chain where the C-terminal truncation mutant is created. In other words, the C-terminus of the Aα chain of fibrinogen is truncated and a protein or peptide species X is attached to the truncated end of the Aα chain. While it may be useful to leave the empty space within the fibrinogen Aα chain, more preferably the space will be filled with an exogenous X domain as a fusion protein. Examples of X include but are not limited to the examples discussed below.

Adhesion domains: Many extracellular matrix molecules and matricellular signal through their adhesion domains, including collagens, laminin, fibronectin, vitronectin, thrombospondins, L1, SPARC family members, elastin, ostopontin, the CCN family, ICAMs, CAMs, dystrophin, dystroglyan, proteoglycans, and so forth. The domains of the proteins that bind to the cell adhesion receptors on cells can often be localized to smaller domains of these proteins. An illustrative example is fibronectin, in which the 9^{th} and 10^{th} type-III repeat domains contain two cell-binding domains that operate alone or in synchrony, namely an RGD site and a PHSRN (SEQ ID NO: 2) site. Thus, in this case, X may be a short peptide comprising the sequence RGD, a short peptide comprising the sequence PHSRN (SEQ ID NO: 2), or both. In preferred embodiments, whole protein domains will be used, allowing the fullness of their evolutionarily-determined structure to be incorporated into the fibrinogen variant. These adhesion domains can be useful for incorporating migration-inducing, angiogenic, and more generally morphogenetic character into fibrin gels formed including the X-containing fibrinogen fusion protein.

Growth factors: Many growth factors signal by binding to cell-surface receptors, including vascular endothelial growth factors, platelet-derived growth factors, fibroblast growth factors, transforming growth factor-betas, insulin-like growth factors, parathyroid hormone, angiopoietin, thrombopoietin, connective tissue growth factor, nerve growth factors, neurotrophins, epidermal growth factor, etc. The above list is only a partial list of the many growth factors that are useful as fibrinogen fusion proteins. Some of the growth factors, such as the fibroblast growth factors, are monomeric, and these can be incorporated directly and without complexity as domains X in a fusion protein. Others, such as vascular endothelial growth factor, are dimeric. In such cases, it may be necessary to incorporate one monomer unit as a domain X in the fusion protein fibrinogen mutant, and to co-express the monomer X (as a soluble protein, not as a domain in the fusion protein) so that this monomer will dimerize with the copy of X that is present in the fusion protein. This would thus involve expressing both X and the X-containing fusion protein, either simultaneously or sequentially. Either full length growth factors or only the receptor-binding domains of these proteins can be incorporated into the fusion protein.

Cytokines and chemokines: Just as growth factors are powerful morphogens, the chemokines and cytokines are powerful cellular regulators and morphogens. Morphogens are signaling molecules that emanate from a restricted region of a tissue and spread away from their source to form a concentration gradient. These include, but are not limited to interleukins, platelet activating factors, CCR molecules, CXC molecules, and many other families of proteins. Either full length proteins or only the binding domains of these proteins can be incorporated into the fusion protein.

Antiadhesion domains: Some proteins function as negative regulators of cell adhesion, repelling rather than inducing cell adhesions. These molecules include domains of thrombospondin, such as the SPAC domain. Antiadhesion domains may be useful in preventing scar formation, in preventing cellular migration and infiltration. Either full length proteins or only the binding domains of these proteins can be incorporated into the fusion protein.

Immunostimulatory and immunomodulatory domains: Some proteins function as immunostimulatory and immunomodulatory molecules. One example is flagellin, a domain of which is known to bind to members of the toll-like receptor family and activate maturation of dendritic cells, leading to more effective antigen presentation and maturation of immune responses. In this case, either the whole protein flagellin or domains of flagellin may be incorporated as X as a domain in a fibrinogen fusion protein. Other proteins of interest include, but are not limited to, bacterial coat proteins, mannose receptor ligands, and viral coat proteins.

Protein-binding domains: Many proteins have evolved binding domains for other proteins. For example, members of the transforming growth factor beta family bind to extracellular matrix proteins such as members of the collagen family. In this case, such domains of collagen may be incorporated into fibrinogen mutants as domains X. Alternatively, protein-binding domains could be identified by computational methods or by combinatorial methods for incorporation as domains X. As a specific example of protein-binding domains, proteins that bind to the extracellular matrix molecules are of particular interest in regenerative medicine, including fibronectin, which binds collagen and thrombospondin; and nidogen, which binds elastins and laminins. Either full length proteins or only the domains of these proteins can be incorporated into the fusion protein.

Nucleic acid-binding domains: Many proteins contain DNA-binding and RNA-binding domains. Such proteins include transcription factors and histone proteins. Moreover, DNA-binding domains can be identified computationally or combinatorially, and oligomers and polymers of lyine, argine, and histidine also bind DNA. Such domains can be incorporated as domains X in fibrinogen fusion proteins, for the purpose of binding do DNA in gene delivery, antisense oligonucleotide delivery, and si-RNA delivery.

Heparin-binding domains: Many proteins contain polysaccharide-binding domains, e.g. those having affinity for heparin, heparin sulfate, chondrointin sulfate, and dermatan sulfate. These domains may be useful to immobilize polysaccharides within fibrin matrices, either because of the active character of the polysaccharide or due to its ability to bind to other proteins.

Virus-binding domains: Some proteins bind to viral coat proteins, e.g. the coxsackie-adenoviral receptor. Incorporation of such virus-binding domains can be accomplished for better retention and delivery of viral vectors in gene delivery.

Cytotoxic domains: Some proteins by to cell-surface receptors and induce cell death via apoptosis. These proteins include the FAS ligand. Incorporation of such domains can be accomplished for prevention of scar formation, cell infiltration and cell migration, and may be useful in the local treatment of tumors.

Enzymatically active domains: Some proteins have enzymatic activity, such as proteases and transglutaminases. These proteins can be incorporated to provide a long-term chemically reactive character to the resulting fibrin gel, including the ability to locally convert pro-drugs to active drugs within the fibrin matrix containing such an enzyme as an X domain in a fibrinogen fusion protein. Proteases incorporated as an X domain may influence fibrin degradation rate, and transglutaminases may incorporate other exogenous proteins within the fibrin network or also influence degradation rate. Either full length proteins or only the binding domains of these proteins can be incorporated into the fusion protein.

Protease inhibitor domains: Some proteins inhibit proteases, and these can be incorporated as X domains within fibrinogen fusion proteins, e.g. to influence degradation rate or the resulting fibrin network or of other matrix proteins co-incorporated within the fibrin matrix. Either full length proteins or only the binding domains of these proteins can be incorporated into the fusion protein.

The above list of examples of proteins that can be incorporated as domains X within fibrinogen fusion proteins, within the space created by forming the Aα truncation mutant, is only an illustrative list. In many cases, it will be possible to incorporate the full-length protein, or smaller protein truncations, or even peptide domains that represent the active domains of these proteins.

### II. Expression of Modified Truncated Fibrinogen

Standard cloning techniques that are well known to one of ordinary skill in the art can be used to generate fusion proteins.

Oligonucleotides for use as primers for amplification and probes for hybridization screening may be designed based on any known DNA sequence. Oligonucleotide primers for amplification of a full-length cDNA are preferably derived from sequences at the 5′ and 3′ ends. Primers for amplification of specific regions are chosen to generate products of a detectable size.

Amplification primers preferably do not have self-complementary sequences nor have complementary sequences at their 3′ end (to prevent primer-dimer formation). Preferably, the primers have a GC content of about 50% and may contain restriction sites to facilitate cloning. Amplification primers usually are at least 15 bases and usually are not longer than 50 bases, although in some circumstances and conditions shorter or longer lengths can be used. Usually, primers are from 17 to 40 bases long, 17 to 35 bases long, or 20 to 30 bases long. The primers are annealed to cDNA or genomic DNA and sufficient amplification cycles, generally 20-40 cycles, are performed to yield a product readily visualized by gel electrophoresis and staining or by hybridization. The amplified fragment can be purified and inserted into a vector and propagated, isolated and subjected to DNA sequence analysis, subjected to hybridization, or the like.

A DNA sequence encoding fibrinogen, a variant, or a fusion protein is introduced into an expression vector appropriate for the host. In certain embodiments, fibrinogen is inserted into a vector such that a fusion protein is produced. A preferred means of synthesis is amplification of the gene from cDNA using a set of primers that flank the coding region or the desired portion of the protein. Restriction sites are typically incorporated into the primer sequences and are chosen with regard to the cloning site of the vector. If necessary, translational initiation and termination codons can be engineered into the primer sequences.

At a minimum, the vector must contain a promoter sequence. Other regulatory sequences may be included. Such sequences include a transcription termination signal sequence, secretion signal sequence, origin of replication, selectable marker, and the like. The regulatory sequences are operationally associated with one another to allow transcription or translation.

The plasmids used herein for expression of fibrinogen fusion proteins include a promoter designed for expression of the proteins in a host cell. Suitable promoters are widely available and are well known in the art. Inducible or constitutive promoters are preferred. Promoters for expression in eukaryotic cells include, but are not limited to, the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009) and other inducible promoters. In a preferred embodiment, the promoter is the elongation factor-1A (EF1A) promoter. For expression of the proteins, a promoter is inserted in operative linkage with the coding region for the fibrinogen fusion protein.

In other preferred embodiments, the vector includes a transcription terminator sequence, which has either a sequence that provides a signal that terminates transcription by the polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the f1-ori arid col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}) tetracycline resistance gene (Tc^{r}) and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance, puromycin resistance and hygromycin resistance).

A wide variety of suitable vectors for expression in eukaryotic cells are available. Such vectors include, but are not limited to, pCMVLacI, pXT1 (Stratagene Cloning Systems, La Jolla, Calif.); pCDNA series, pREP series, pEBVHis (Invitrogen, Carlsbad, Calif.). Suitable eukaryotic cells include yeast, insect, and mammalian cells.

In a preferred embodiment, mRNA is isolated from human cells and is subjected to reverse transcription to generate complementary DNA (cDNA). PCR primers are designed to contain restriction enzyme sites and to hybridize to the 5′ and 3′ end of fibrinogen cDNA accordingly. It will be well understood by one of ordinary skill in the art that the 3′ PCR primer will vary according to the desired truncated fibrinogen product to be produced. The PCR products are purified, digested with restriction enzymes and ligated into an expression vector. Preferably, following ligation of the truncated fibrinogen into the vector, a multiple cloning site (MCS) is located at the 3′ end of the truncated fibrinogen.

Using these templates, fibrinogen Aα chain can be fused to any protein or peptide-based receptor ligand and/or protein/DNA binding partner at the carboxy-terminus of the chain using standard cloning techniques to generate a cDNA product that can be ligated into the provided MCS such that the added cDNA sequence is “in frame” with respect to preceding sequence encoding the fibrinogen Aα chain. The term “in frame” refers to the orientation of the DNA translation codons such that the fusion protein is translated appropriately. The “in frame” nature of the resulting Aα-X transgene (where X is the cDNA encoding any protein/peptide motif) is an absolute requirement to generate a full length Aα fusion protein product. The newly created vector in conjunction with vectors encoding the fibrinogen Bβ (Accession #NM_{—}005141) and γ (Var A, NM_{—}000509 and Var B. NM_{—}021870) chains are co-transfected (a process to incorporate DNA into cells) into any mammalian cell line with all three fibrinogen chains. Transfection of DNA into cells can be achieved by any of the well known methods in the art. Cells can be transfected in a transient or stable (via selection with puromycin antibiotic) manner. Stable transfection cell clones can be established via standard techniques. The secreted nature of fibrinogen results in protein product in the supernatant of the cell culture. Following sufficient transfection of a transient culture or expansion of stable cell lines sufficient quantities of fibrinogen can be produced, depending on the conditions of culture and the cell line chosen as the bioreactor, for protein purification.

### III. Purification of Modified Truncated Fibrinogen Fusion Protein Products

The vector encoding the Aα protein fusion in conjunction with vectors encoding the fibrinogen Bβ and γ chains are co-transfected into a mammalian cell line. The secreted nature of fibrinogen results in protein product in the supernatant of the cell culture. Following transfection of a transient culture or expansion of stable cell lines sufficient quantities of fibrinogen can be produced, depending on the conditions of culture and the cell line chosen as the bioreactor, for protein purification.

Fibrinogen fusion protein products can be purified by any number of established means including precipitation or size or affinity column purification. In one embodiment, purification is carried out by affinity chromatography with a peptide affinity resin consisting of the peptide GPRPAA tethered to a Fractogel® column. It is understood by one of skill in the art that any solid substrate matrix will suffice. Fibrinogen molecules bind, in a specific manner, via a domain in the C-terminal region of the γ chain (termed “A-hole”) to this peptide sequence under physiologic conditions, in a preferred embodiment 0.1 M HEPES buffer containing 20 mM CaCl₂. Fibrinogen Aα fusion proteins do not interfere with this purification technique since the active binding site used is located in an adjacent polypeptide chain. Fibrinogen molecules are then eluted from the affinity resin under mildly acidic conditions, specifically 1 M NaBr solution containing 50 mM NaAc at pH 5.3. Following elution the fibrinogen must be rapidly reequilibrated into a buffer with physiologic salt and pH values, in a preferred embodiment 50 mM Tris, 150 mM NaCl, pH 7.2. Purification under these conditions yields a highly purified fibrinogen solution that retains native capacity to form a crosslinked polymer.

### IV. Formulations of Fibrinogen Aα Fusion Proteins and Uses Thereof.

Purified fibrinogen Aα fusion proteins have several formulations for use including as a soluble protein species and in polymer form following the addition of an activator, in most cases the protein thrombin. As a soluble protein, fibrinogen fusion proteins may be mixed with carrier proteins that serve a protective role or proteins that interact with the fibrinogen fusion protein in a specific way. In one embodiment, the fibrinogen fusion proteins are activated to form fibrin polymer. In a preferred embodiment, fibrinogen fusion proteins are mixed with native fibrinogen to form a mixture for the generation of fibrin polymer. It is understood by one of ordinary skill in the art that the conditions of the mixture can vary and will depend on the therapeutic dose of the generated fibrinogen fusion proteins. Additional additives to this basic formulation include Factor XIII (or Factor XIIIa), pH buffers, anti-proteolytic agents, and other chemicals/biochemical species that interact with the fibrinogen fusion protein in a specific way (i.e. plasmid DNA with a DNA-binding fibrinogen Aα fusion protein).

Fibrinogen fusion proteins are useful for enhancing the incorporation of a therapeutic protein or peptide species into a fibrin polymer for sustained presentation of such therapeutics. The therapeutic protein/peptide species include, but are not limited to, receptor ligands such as growth factors and cell adhesion molecules and soluble protein or nucleic acid binding domains. The use of fibrinogen fusion proteins as a fibrin-based therapeutic delivery system simplifies the mode of therapeutic incorporation by coupling, at the genetic level, the elements of fibrinogen that allow polymer formation and a deliverable protein species. The advantage of this is that all of the deliverable protein species are incorporated without additional steps beyond the simple polymerization of the fibrinogen/fibrin system via thrombin activation.

These materials may be useful in the promotion of healing and tissue regeneration, in the creation of neovascular beds for cell transplantation and in other aspects of tissue engineering.

Following polymerization, the fibrinogen fusion proteins may take the form of a porous vascular graft, such as a scaffold for skin, bone, nerve or other cell growth. Additionally, the polymerized fibrinogen fusion proteins may be used as surgical sealants or adhesives.

The fibrinogen fusion proteins can also be used in methods for promoting cell growth or tissue regeneration. This method involves producing a fibrin comprised solely of fibrinogen fusion proteins or a mixture of native fibrinogen and the fibrinogen fusion proteins and exposing the fibrin to cells or tissue to promote cell growth or tissue regeneration. This method may be used in conjunction with a variety of different cell types and tissue types. Such cell types include, but are not limited to, nerve cells, skin cells, and bone cells.

The present invention will be further understood by the following non-limiting examples.

### EXAMPLES

### Example 1

### Generation of Fibrinogen Aα Fusion Protein Products

The two base inserts described herein are 1) the full length homo sapiens fibrinogen Aα chain DNA sequence minus the original stop codon (NCBI Accession number NM_{—}021871, base 31 through 1962 (amino acids 1 to 644) and 2) a model truncation of fibrinogen Aα chain corresponding to bases 31 thru 597 (amino acids 1 through 189). A multiple cloning site (NCS) consisting of EcoRV, NotI, EcoRI, ClaI, and NheI was constructed immediately adjacent to these DNA sequences by the following standard cloning strategy.

First, mRNA was isolated from human liver cells (HepG2) and subjected to reverse transcription to generate complementary DNA (cDNA). The above base inserts were amplified from the HepG2 cDNA by polymerase chain reaction (PCR) using the 5′ primer-CAGCCACTAGTTTAGAAAAGATGTTTT (SEQ ID NO: 3) for both products and the 3′ primers-GGGCCCTCTAGAGATATCTTAGTCTAGGGGGACA (SEQ ID NO: 4) and GGGCCCTCTAGAGATATCAGCTAAAGCCCTACT (SEQ ID NO: 5) for the fall length and truncated products, respectively.

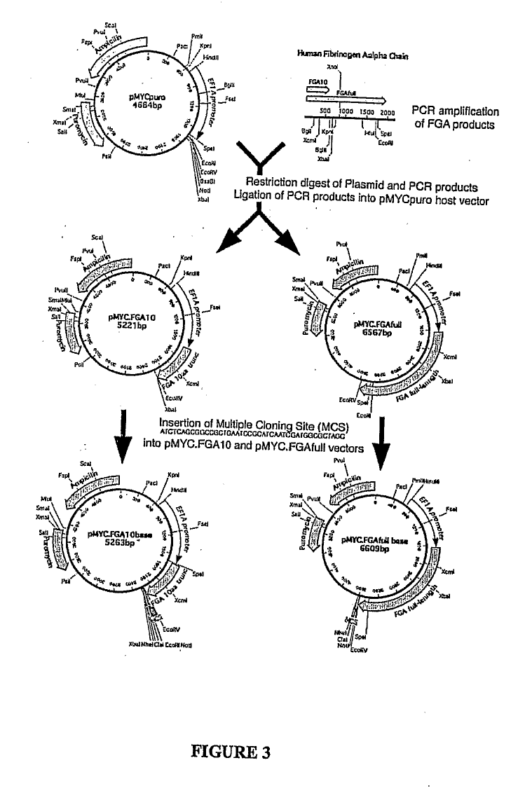

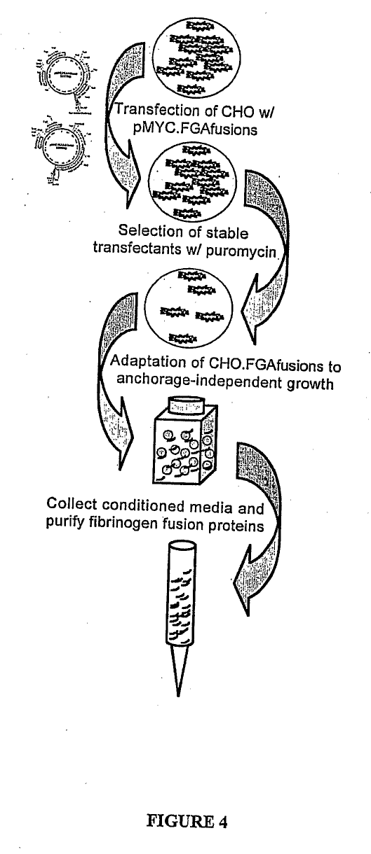

The subsequent PCR products were purified, digested with SpeI and XbaI restriction enzymes and ligated into the SpeI and XbaI sites of an expression vector containing the EF1A promoter driving the fibrinogen full length and truncation transgenes and containing the antibiotic resistance genes ampilicin and puromycin, although those skilled in the art will acknowledge that the choice of expression vector will have no effect on the transgene product produced. The resultant purified vectors (termed pMYC.FGAfull and pMYC.FGA10, respectively) were subsequently digested with the restriction enzyme EcoRV and the double-stranded oligonucleotide ATCTCAGCGGCCG CTGAATTCGCATCAATCGATGGC GCTAGC (5′-3′ sequence) (SEQ ID NO: 6) ligated into the EcoRV site resulting in vectors pMYC.FGAfull base and pMYC.FGA10 base, respectively. The resultant vectors correspond to the described “base inserts” within the context of the pMYCpuro vector system. A schematic diagram of the cloning strategy is shown in FIG. 3.

Using the above template fibrinogen Aα chain vectors (pMYC.FGAfall base and pMYC.FGA10 base) any protein or peptide-based receptor ligand and/or protein/DNA binding partner can be fused to the base fibrinogen Aα chain by using standard cloning techniques to generate a cDNA product that can be ligated into the provided MCS such that the added cDNA sequence is “in frame” with respect to preceding sequence encoding the fibrinogen Aα chain. The “in frame” nature of the resulting Aα-X transgene (where X is the cDNA encoding any protein/peptide motif) is an absolute requirement to generate a full length Aα fusion protein product. The newly created vector in conjunction with vectors encoding the fibrinogen Bβ (Accession #NM_{—}005141) and γ (Var A, NM_{—}000509 and Var B. NM_{—}021870) chains are co-transfected (a process to incorporate DNA into cells) into CHO cells, or any mammalian cell line, using any of the established techniques with all three fibrinogen chains. Cells can be transfected in a transient or stable (via selection with puromycin antibiotic) manner, or stable cell clones established via standard techniques. The secreted nature of fibrinogen results in protein product in the supernatant of the cell culture. Following sufficient transfection of a transient culture or expansion of stable cell lines sufficient quantities of fibrinogen can be produced, depending on the conditions of culture and the cell line chosen as the bioreactor, for protein purification.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims. Those skilled in the art will recognize, or be able to ascertain using no more than routine experimentation, many equivalents to the specific embodiments of the invention described herein. Such equivalents are intended to be encompassed by the following claims.

1. A nucleic acid encoding
a truncated fibrinogen Aα chain, wherein the fibrinogen Aα chain contains a truncation site at its carboxy terminus C-terminal to amino acid residue 179; and
a non-fibrinogen bioactive factor C-terminally attached to the truncation site, wherein the non-fibrinogen bioactive factor is selected from the group consisting of adhesion proteins, growth factors, cytokines, chemokines, antiadhesion proteins, immunostimulatory proteins, immunomodulatory proteins, protein-binding proteins, nucleic acid-binding proteins, heparin-binding proteins, virus-binding proteins, cytotoxic proteins, enzymatically active proteins, and protease inhibitors.

2. The nucleic acid of claim 1 wherein the Aα chain contains a truncation site at its carboxy terminus C-terminal to amino acid residue 184.

3. The nucleic acid of claim 1 wherein the Aα chain contains a truncation site at its carboxy terminus C-terminal to amino acid residue 189.

4. (canceled)

5. The nucleic acid of claim 4 wherein the bioactive factor is a protein or a domain of a protein.

6. (canceled)

7. The nucleic acid of claim 1 wherein the fibrinogen is human fibrinogen.

8. The nucleic acid of claim 7 wherein the fibrinogen contains a conservative substitution, addition or deletion not substantially affecting function or structure.

9. An expression vector comprising the nucleic acid of claim 2.

10. A method of making a fibrinogen fusion protein comprising
a) expressing a nucleic acid encoding a truncated fibrinogen Aα chain and a non-fibrinogen bioactive factor C-terminally attached to a truncation site as selected from the group consisting of C-terminal amino acid residue 179, 184, and 189.

11. The method of claim 10 further comprising
b) transfecting a host cell with an expression vector encoding the non-fibrinogen bioactive factor inserted into the truncation site of the fibrinogen Aα chain selected from the group consisting of C-terminal amino acid residue 179, 184, and 189, the Bβ chain of fibrinogen and the γ chain of fibrinogen, wherein the host cell expresses the Aα fusion chain, the Bβ chain and the γ chain; and
c) isolating the fibrinogen fusion protein.

12. The method of claim 11 wherein the fibrinogen fusion protein, the Bβ chain and the γ chain are on two or more separate vectors.

13. The method of claim 12 wherein the vectors are co-transfected into a host cell.

14. A fibrinogen fusion protein expressed from the nucleic acid of claim 1.


SEQUENCE LISTING (6 sequences)

SEQ ID NO: 1 - protein, 8 aa, Homo sapiens
NQEQVSPL

SEQ ID NO: 2 - protein, 5 aa, Homo sapiens
PHSRN

SEQ ID NO: 3 - DNA, 27 bp, Artificial Sequence
  5′ Primer for fibrinogen A alpha chain
cagccactagtttagaaaagatgtttt

SEQ ID NO: 4 - DNA, 34 bp, Artificial Sequence
  3′ Primer for full length fbrinigen A alpha chain
gggccctctagagatatcttagtctagggggaca

SEQ ID NO: 5 - DNA, 33 bp, Artificial Sequence
  3′ Primer for truncated fibrinogen A alpha chain
gggccctctagagatatcagctaaagccctact

SEQ ID NO: 6 - DNA, 42 bp, Artificial Sequence
  Double Stranded Oligonucleotide
atctcagcggccgctgaattcgcatcaatcgatggcgctagc